(19) United States
(12) Patent Application Publication
(10) Pub. No.: US 2004/0082014 A1
(43) Pub. Date: Apr. 29, 2004

(54) Method and test system for identifying substances which protect nerve cells

(76) Inventors: Michael Sendtner, Veitshochheim (DE); Ulf Rudiger Rapp, Wurzburg (DE); Stefan Wiese, Wurzburg (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/470,068

(22) PCT Filed: Jan. 22, 2002
(86) PCT No.: PCT/EP02/00590

(30) Foreign Application Priority Data
Jan. 22, 2001 (DE) 101027222

(51) Int. Cl. A61K 31/00

(52) U.S. Cl. 435/7.2; 435/7.21; 514/1; 514/2

(57) ABSTRACT

The invention relates to a method for identifying pharmacologically active ingredients which influence the function of cells in the central nervous system. The inventive method comprises the following steps: a) a sample is brought into contact with at least one potential active ingredient, and b) the activity of Raf, especially B-Raf, in the sample is determined.

### BACKGROUND OF THE INVENTION

The subject matter of the present invention is a method for identifying pharmacologically active ingredients which influence the function of cells in a central nervous system, comprising the following steps: a) a sample is brought into contact with at least one potential active ingredient, and b) the activity of Raf, especially B-Raf, in the sample is determined.

Although differentiated neurons belong to the longest living cell types in mammals, undifferentiated nerve cells die in large numbers during the development of the nervous system. Further, the death of neuronal cells is a substantial feature of acute and chronic neurodegenerative diseases. The question of how nerve cells die, is not yet fully understood in all details.

It is however known that nerve cells, similar to all other cells, need a trophic assistance for their survival.

It is also known that nerve cells, irrespective of whether they have a sensor or a motor function, can only survive in presence of a number of so-called neurotrophic factors. These neurotrophic factors are members of different families. To them belong the neurotrophins, such as the brain derived neurotrophic factor (BDNF), the ciliary neurotrophic factor (CNTF), neutrophin-3 (NT-3) and the nerve growth factor (NGF) (Kaplan and Miller, Curr. Opin. Neurobiol. 10:381-291, 2000), the hepatocyte growth factor (HGF) (Maina and Klein, Nat. Neurosci. 2:213-217, 1999) and the glial cell derived neurotrophic factor (GDNF) (Balok et al., Curr. Opin. Neurobiol. 10:103-110, 2000).

Such neurotrophic factors act by the binding to and activation of tyrosine phosphokinase receptors. From these receptors, the activation signal is transferred by signal transducing proteins present in the cytoplasm into the cell core. Several signal transduction pathways are known in neurons. To these belongs the PI-3K-AKT signal transduction pathway and the Ras-Raf transduction pathway. Both activation pathways are cross-linked by activated RAS, a protein playing a key role in the Raf transduction pathway (Yuan and Yankner, Nature 407:802-809, 2000). Additional Raf-dependent signal transduction pathways are the Bag-1-C-Raf signal pathway (Wang et al., PNAS USA 93:7063-7068, 1996) and the Rap-1-B-Raf-AMP signal pathway (Grewal et al., J. Biol. Chem. 275:3722-3728, 2000).

An essential result of the activation of neuronal cells and glial cells by neurotrophic factors is the increased expression of intracellular proteins protecting the cells from a controlled cell death (Newmeyer and Green, Neuron 21:653-655, 1998; Wiese et al., Nature Neurosci. 2:978-893, 1999). To these proteins belong the inhibitors of the IAP/ITA family, in particular IAP-1, IAP-2, x-IAP and survivin. Proteins of the IAP/ITA family inhibit the activation of procaspase-9 (Deveraux et al., EMBO J. 17:2215-2223(1998)), which in turn is activated by cytochrome-C and Apaf-1. Further, proteins of the IAP/ITA family inhibit the function of the activated caspases-3, -6 and -7 and may also thereby inhibit the apoptosis caused by these enzymes (Devereaux et al., Nature 388:300-304, 1997; Roy et al., EMBO J. 16:6914-6925, 1997).

Akt has three cellular isomers, of which Akt-3 is expressed particularly in neurons (Datta et al., Genes Dev. 13:2905-2927, 1999). For the Raf signal transduction pathway in mammalian cells, three different Raf proteins play a special role: C-Raf (also called Raf-1), A-Raf and B-Raf. Several forms caused by different splicing exist for B-Raf. In cells of the central nervous system, mainly B-Raf and C-Raf protein can be detected, A-Raf protein to a smaller degree (Morice et al., Eur. J. Neurosci. 11:1995-2006, 1999)

B-Raf and C-Raf cannot only be detected in neurons, but also in glial cells (Mikaly et al., Brain Res. 27:225-238, 1993; Mikaly and Rapp, Acta Histochem. 96:155-164, 1994).

Although it is relatively clear how neurotrophins and/or membrane depolarization activate signal transduction pathways securing the survival of nerve cells, it is still open which mechanisms lead to the death of nerve cells in the case of neurodegenerative diseases. Experimental investigations (a survey can be found in Yuan and Yankner, Nature 407:802-809, 2000) indicate that the death of nerve cells can be initiated by

the relative activation of proapoptotic factors within the nerve cell, for instance by lacking phosphorylation and inactivation of proapoptotic proteins (Bad, in particular however Bax) for the AKT and/or Raf signal transduction pathways,

the lacking activation (for instance for the Raf signal transduction pathway) of transcription factors activating the transcription of genes expressing proapoptotic proteins (e.g. Bcl-2, in particular however Bcl-XL),

the damage to mitochondria with the release of cytochrome C and the activation of proapoptotic enzymes (caspases) for instance by abnormal protein structures or aggregates,

by the activation of proapoptotic signal cascades by neurotrophins for instance by means of the neurotrophin receptor p75 NTR,

by oxidative damages, NO overproduction or by enzymatic malfunctions, e.g. by mutations of the superoxide dismutase (SOD).

In consideration of these diverse possibilities for the death of nerve cells, it has been extremely difficult up to now to establish model systems, by means of which substances inhibiting the dying process of nerve cells can specifically be detected and tested.

### GENERAL DESCRIPTION OF THE INVENTION

Surprisingly it has been found that the survival of sensor and motor neurons caused by neurotrophic factors depends from the intracellular presence of the signal protein B-Raf. Nerve cells without B-Raf will die in spite of the presence of neurotrophic factors. In contrast thereto, nerve cells expressing B-Raf, but not C-Raf, will survive in the presence of neurotrophic factors, same as nerve cells comprising B-Raf as well as C-Raf (so-called normal (=wild-type) nerve cells). The basis of the present invention is thus the surprising finding that a B-Raf able to function (i.e. enzymatically active) is necessary for the survival of nerve cells. Furthermore, there is a surprising finding that in nerve cells with lacking B-Raf activity, the expression of the anti-apoptotic proteins of the IAP/ITA family, for instance of IAP-1, IAP-2 and x-IAP, is clearly reduced.

The subject matter of the invention is thus a method for identifying pharmacologically active ingredients which influence the function of cells in a central nervous system, comprising the following steps: a) a sample is brought into contact with at least one potential active ingredient, and b) the activity of Raf, especially B-Raf, in the sample is determined.

As the “functions” of cells of the central nervous system are designated for instance the conduction of stimulation and any other involved biochemical and/or electro-chemical processes. Furthermore, the term function of cells also comprises the survival of the cells. The death of cells of the central nervous system may for instance be observed by test methods detecting the apoptosis. Such test methods are for instance the “tunnel assay” (Gavrielli et al., J. Cell Biol., 119:493-501, 1992, Gold et al., Lab. Invest. 71:219-225, 1994), the chromatin fragmentation (Götz et al., Hum. Mol. Genet. 9:24792489, 2000), the counting of surviving and dying nerve cells (Arakawa et al., J. Neurosci. 10, 3507-3515, 1990), the use of test substances for quantifying the cell death in cell culture (Uliasz and Hewett, J. Neurosci. Methods 100, 157163, 2000), the quantification of the expression of cell death-associated genes in nerve cells, such as cyline (Timsit et al., Eur. J. Neurosci. 11, 263-278, 1999) and the determination of the neuronal cell death after addition of Aβ (Iwasaki et al., Mol. Psych. 1, 65-71, 1996) or after the induction of oxidative stress (Manev et al., Exp. Neurol. 133, 198-206, 1995).

Cells of the central nervous system in the meaning of the present invention are glial cells or neuronal cells, for instance sensor and sympathic neuronal cells, motor neuronal cells, cholinergic neurons in the basal forebrain, dopaminergic nerve cells of the midbrain (substantia nigra), granule cells and Purkinje cells of the cerebellum and the hippocampus, retinal ganglion cells and photoreceptors as well as neuronal stem cells.

Bringing a sample into contact with at least one potential active ingredient comprises for instance any form of mixing, and the sample may be added to the potential ingredient, or the potential ingredient to the sample. The sample and/or the potential ingredient may be provided as a solid matter, solution, suspension, flotation or bound to a solid phase. If the sample with which the potential ingredient(s) is (are) brought into contact, are cells, then the step of bringing into contact also comprises prior art methods permitting the introduction of substances into intact cells, such as infection, transfection and/or transformation. These methods are particularly preferred, if the potential ingredient is naked DNA, viruses, virosomes and/or liposomes, the liposomes or virosomes being also suited to bring further potential ingredients into contact with the sample, in addition to a potentially active nucleic acid molecule. A number of further methods are known to the man skilled in the art, such methods serving for the introduction of potential active ingredients into cells.

A potential active ingredient in the meaning of the present invention may be any molecular species, such as for instance a peptide (between 1 to 50 amino acids), a protein (more than 50 amino acids), a peptoid, an oligo or polysaccharide, a nucleic acid, a monomer such as for instance a homo or heterocycle, a lipid, a steroid and the like. Basically, any chemical substance or mixture of substances may be a potential active ingredient to be used in the method according to the invention. The concentration of the potential active ingredient has however to be selected such that the influence on the activity of Raf, in particular B-Raf, in the sample is not simply based on the lysis of the cells, if the sample is a cell, or on the denaturation of Raf, in particular B-Raf, if the sample is a protein or a mixture of proteins. Accordingly are for instance guanidin-HCl solution, urea solution and strong detergents in concentrations at which they lysate cells and/or denaturate proteins, no potential active ingredients in the sense of the present invention.

A sample in the sense of the present invention is at least one cell, at least cell extract, at least one protein mixture and/or at least one mixture containing Raf protein, in particular B-Raf or activated B-Raf, or a part thereof. These cells comprise for instance pro and eukaryotic cells, in particular cells which as wild-type cells express Raf, in particular B-Raf. In cells which as wild-type cells do not or only to a small degree express Raf, Raf protein can be expressed by methods known to the man skilled in the art. Such methods comprise for instance infection, transfection or transformation of cells with vectors containing nucleic acids coding for Raf, in particular B-Raf, or parts thereof. A preferred sample which can be used in the method according to the invention is a cell having a reduced or no Raf activity, in particular B-Raf activity, at all. Such a cell can for instance be obtained from heterozygous or homozygous Raf knock-out mice. Such cells are then for instance for a-raf, b-raf or c/raf (−/−) or (+/−). Cells preferred in the method according to the invention are neurons or neuronal stem cells obtained from heterozygous or homozygous Raf knock-out mice or mouse embryos. Such cells can for instance be obtained from b-raf (−/−) deficient mice (Wojnowski et al., Nature Genetics 16:293-297, 1997).

Further the term sample also comprises cell extracts which can for instance be obtained from one of the above cells by standard methods known to the man skilled in the art; suitable methods are however not limited to “freeze thawing”, “sonification” or “French pressing”. If applicable, such a cell extract can be processed or purified in further steps. Preferred steps comprise for instance precipitation, filtration and chromatographic steps. Suitable chromatographic steps are known to the man skilled in the art and comprise for instance anion or cation exchange chromatography, affinity chromatography and/or size exclusion chromatography. Further, the sample may also be a mixture of purified or recombinant proteins containing Raf, in particular B-Raf, and/or a protein mixture which contains further components, for instance components which can be used for the determination of the activity of B-Raf, such as for instance substrates of Raf, buffer, detergents, protease inhibitors NTPs and/or suitable metal ions. The Raf protein included in the sample may be B-Raf protein, C-Raf protein and if applicable also A-Raf protein, in particular however B-Raf protein. Preferably, the Raf protein included in the sample is an activated Raf protein, i.e. it has a higher serine/threonine phosphokinase activity compared to the wt Raf protein. Raf protein is for instance activated by a reversible phosphorylation. A constitutive activation is however also possible by the introduction of mutations, suitable mutations being related for instance to the N-terminal section of the enzyme, in particular in C-Raf-1 the mutations of ²⁵⁹Ser to ²⁵⁹Ala and the mutation of the analog positions in B-Raf or mutations within the CR2 region, insertion of linker structures in this region or deletion of the complete N-terminus of Raf-1 (Daum et al., TIBS 19, 474-480; Morrison and Cutler, Curr. Op. Cell. Biol. 9, 174-179, 1997).

Determining the activity of Raf in the sample is possible by a number of direct and indirect detection methods. The respectively suitable methods depend on the nature of the sample. In cells, the activity of Raf is on the one hand determined by the amount of the Raf expressed in the cell, and on the other hand by the amount of the activated Raf. The activation of the transcription of the genes coding for Raf protein, in particular B-Raf protein, may for instance be made by determining the amount of the Raf mRNA. Prior art standard methods comprise for instance the DNA chip hybridization, RT-PCR, primer extension and RNA protection. Furthermore, the determination of the Raf activity based on the induction or repression of the transcription of the respective Raf gene(s), may also take place by the coupling of the Raf promoter to suitable reporter gene constructs. Examples for suitable reporter genes are the chloramphenicol transferase gene, the green fluorescent protein (GFP) and variants thereof, the luciferase gene and the Renilla gene. The detection of the increase of expression of Raf proteins may however also be made on the protein level, in this case the amount of protein being detected for instance by antibodies directed against Raf protein. The change of the activity of the Raf protein can however also be put down to increased or reduced phosphorylation or dephosphorylation of the protein. For instance, the B-Raf kinase is regulated by the phosphorylation of the ⁵⁹⁸Thr and ⁶⁰¹Ser remainders (Zhang B. H. and Guan K. L. EMBO J. 19:5429, 2000). The change of the phosphorylation of B-Raf proteins may for instance be detected by antibodies directed against phosphorylated threonine or serine.

Since Raf proteins are threonine/serine kinases, the activity of the Raf proteins can also be determined by their enzymatic activity. The protein MEK is for instance a substrate of B-Raf and the degree of the phosphorylation of MEK permits the determination of the B-Raf activity in the sample. In the same way, the phosphorylation of other substrates (as for instance MBP and peptides which are specifically phosphorylated by Raf (Salh et al., Anticancer Res. 19, 731-740, 1999, Bondzi et al., Oncogene 19, 5030-5033, 2000)) of the Raf proteins can be used for determining the respective activity. Since Raf is part of a signal cascade where a series of kinases are respectively phosphorylated and activated by a superordinated kinase, the activity of Raf can also be determined by evaluating the phosphorylation degree of each kinase subordinated to Raf. This so-called map kinase pathway leads, among other features, also to a specific activation of transcription factors and thus to a transcriptional activation of genes, such that the activity of Raf can indirectly be determined by measuring the activity of these target genes. To such target genes belong for instance genes which code for the family of the IAP/ITA proteins. Thus, the determination of the activity of Raf may also take place by the determination of the activation of IAP/ITA proteins, in particular the activation of IAP-1, IAP-2, x-IAP and survivin. For the determination of the activation of the target genes, the above methods are suitable.

If the sample is for instance a cell extract, a protein mixture and/or a mixture containing Raf, in particular B-Raf, or a part thereof, the determination of the activity is mainly directed to the determination of the modification of the Raf protein itself or the change resulting thereof of the enzymatic activity of the Raf protein, using the above methods. Preferred methods comprise the determination of the phosphorylation of the immediate substrates of Raf, such as MEK, here the integration of ³²P in MEK or the phosphorylation being possible by an activation-specific MEK antibody which detects phosphorylated MEK only (Bondzi C. et al., Oncogene 19:5030-5033, 2000). Another possibility is for instance the use of a coupled assay using the signal transduction cascade described above and measures the activity of Raf by means of the phosphorylation of substrates subordinated to Raf, such as basic myelin (Bondzi C. et al., Oncogene 19:5030-5033, 2000).

Potential active ingredients enhancing or inhibiting the activity of Raf, in particular B-Raf, in the sample compared to the untreated sample (control) are according to the present invention pharmacologically active ingredients which influence the function of cells in the central nervous system. A pharmacologically active ingredient which influences the function of cells in the central nervous system changes the activity of Raf compared to the control by more than 10%, preferably however by at least 50%, by at least 100%, even more preferred by at least 500%.

In another embodiment an incubation period may follow the step a), and such incubation period may be differently long, in dependence of the sample. If the sample contains cells, the activity (step b) is determined after 1 hour to 100 days, preferably after 1 day to 50 days, even more preferably after 3 day to 10 days, in particular after 3 days. If the sample does not contain cells, the activity can for instance be determined in a time span of approx. 0 seconds (measurement of the activity immediately at bringing into contact) to 20 days. Preferably, the time span for the incubation after bringing the sample into contact with the potential active ingredient is however 5, 10, 20, 30, 40, 50, 60, 90, 120, 150 or 180 min (McDonald et al., Analyt. Biochem. 268, 318-329, 1999).

In a preferred embodiment of the method according to the invention, the sample contains at least one cell, at least one cell extract, at least one protein mixture and/or a mixture containing Raf, in particular activated Raf or a part thereof. A Raf protein part suitable for carrying-out the method according to the invention can still be phosphorylated and/or can act as a series and/or threonine kinase on the respective substrate, such as for instance MEK. The determination of a suitable part of Raf protein is possible by using for instance MEK as a Raf substrate or Raf kinase kinase (Kinuya M. et al. (2000) Biol. Pharm Bull. 23:1158-62) for the phosphorylation of Raf with standard methods.

In a preferred embodiment of the method according to the invention, the cell is a glial cell or a neuronal cell, in particular a sensor neuronal cell, a motor neuronal cell, a neuronal stem cell or a neuron, a neuron to be used in the method according to the invention being for instance differentiable from neuronal stem cells in a cell culture (Vescovi and Snyder, Brain Pathol. 9, 569-598,1999).

In an embodiment of the method according to the invention, the activity of Raf in the cell is determined by the change of the survival rate of the cell. This is of particular interest for cells which have, for instance caused by a mutation, a reduced Raf activity or none at all and which thus have in presence or absence of neurotrophic factors a reduced survival rate compared to the respective wt cells. In particular cells being for b-raf (−/−) have even in presence of neurotrophic factors a significantly reduced survival rate compared to wt cells. An increase of the survival rate of these cells after incubation with at least one potential active ingredient serves as an indirect means of the determination of the activity of Raf.

In a preferred embodiment of the method according to the invention, sensor and/or spinal, motor neuronal cells from b-raf (−/−) or c-raf (−/−) deficient mouse embryos each of the pairing of b-raf or c-raf heterozygous mice are used. Furthermore, from these mouse embryos, neural stem cells can be isolated from the brain and the spinal cord, propagated in cell culture and differentiated to nerve cells. The addition of suitable neurotrophic factors (for instance GDNF, BDNF, CNTF to motor neurons and NGF to sensor neurons) will lead to a survival of c-raf deficient nerve cells, not however to a survival of b-raf deficient nerve cells. Similar investigations can be made with nerve cells which were isolated from neural stem cells of b-raf (−/−) and/or c-raf (−/−) mice.

In an embodiment of the method according to the invention the same potential active ingredient(s) is (are) respectively brought into contact on one hand with c-raf (−/−) deficient cells and on the other hand with b-raf (−/−) deficient cells, and it is determined whether in presence of this (these) test substance(s) b-raf (−/−) deficient nerve cells will survive.

This indirect determination also permits to identify pharmacologically active substances which act in cells having a reduced Raf activity or without detectable Raf activity in a signal transduction reaction subordinated to the Raf kinase.

In a preferred embodiment of the method according to the invention the activity of Raf in the sample is directly or indirectly determined by the amount of the Raf protein, the amount of the nucleic acids coding for Raf and/or the enzymatic activity of Raf. Suitable methods have already been described above.

In another embodiment of the invention the same potential active ingredients(s) is (are) respectively brought into contact on the one hand with a cell extract or with a protein mixture containing C-Raf or with purified or with recombinant C-Raf and on the other hand with a cell extract or a protein mixture containing B-Raf or with purified or with recombinant B-Raf, and the respective activity of C-Raf and B-Raf is determined. A preferred pharmacologically active ingredient influences the activity of B-Raf to a higher degree than the activity of C-Raf. A stronger influence exists if the effect on the activity is at least approx. 2 times, more preferably approx. 4 times, in particular approx. 10 times higher than the effect on the activity of C-Raf.

Another subject matter of the invention is a method wherein in a further step the activity of IAP-1, IAP-2, x-IAP and/or survivin in the sample is determined. Preferably in this method the sample is a cell. The determination of the activity and/or amount of IAP-1, IAP-2, x-IAP and/or survivin can be performed on the protein level by antibodies and/or on the nucleic acid level, as explained above.

In another embodiment of the invention, the sample is compartmentalized, for instance on a microtiter plate with 96, 348 or 1,552 wells. Such microtiter plates are already used as routine in fully automatic, massive parallel test methods permitting to test hundreds of thousands of different potential active ingredients in a short time. Basically every compartmentalization is suitable which permits to spatially limit the effect of the potential active ingredient brought into contact with the sample, such that the effects of the respectively used potential active ingredient on the activity of Raf, in particular B-Raf, in the sample can be determined. The sample may be covalent or non-covalent with the surface of the sample carrier, such as for instance a microtiter plate, may be linked or be present in a solution, a suspension or a flotation. In addition to the prior art microtiter formats being suitable for carrying out the method according to the invention, planar sample carriers or sample carriers having structures of depressions or channels are also suitable. The sample carrier may for instance be made of glass, silicone, metal or plastic.

In another embodiment of the method according to the invention at least one potential active ingredient is covalently or non-covalently linked to a sample carrier, the surface of the sample carrier preferably having a structure in the form of depressions, channels or also being planar. The sample is then brought into contact with the immobilized potential active ingredient, and the activity of Raf, in particular of B-Raf, in the sample is determined at the respective immobilization spot of the potential active ingredient(s). For instance, with protein chips produced by standard methods, for instance known from WO 89/10977, WO 90/15070, WO 95/35505 and U.S. Pat. No. 5,744,305, a protein chip can be produced containing at its surface different peptide fragments, the influence of which on the activity of for instance Raf protein, preferably purified B-Raf protein, can be tested. In the same way, by prior art combinatorial-chemical methods, a plurality of different chemical substances can be produced on a surface, and the effects of such substances on the activity of Raf, in particular B-Raf, can be examined by the method according to the invention.

Another embodiment of the method according to the invention is a method, wherein one or more further steps follow the determination of the activity of B-Raf on the sample, in such steps the pharmacologically active ingredient being isolated. This is of interest in particular when the potential active ingredient is a mixture of active ingredients, as they are for instance found in plant extracts or extracts from microorganisms. The further step(s) which can be used in order to isolate a pharmacologically active ingredient from a complex substance mixture, are known in the art. These methods comprise for instance precipitation, crystallization, chromatographic and separation methods, which are for instance based on the differential solubility of the individual components in different solvents. After every isolation step, the effectivity of the active ingredient can again take place by bringing it into contact with a sample and determining the activity of Raf in the sample.

In another embodiment of the method according to the invention the pharmacologically active ingredient is confected in another step.

After a pharmacologically active ingredient has been determined and/or isolated according to the method according to the invention, this pharmacologically active ingredient can be modified by methods known to the man skilled in the art, such methods comprising for instance the modification with halogens, in particular fluorine or chlorine, and/or combinatorial-chemical approaches, and again be investigated in the method according to the invention, the activity of Raf in the sample of the modified pharmacologically active ingredient being compared to the activity of Raf in the sample when using the original active ingredient.

Another subject matter of the invention is thus also a pharmacologically active ingredient being identified by one of the above methods. Particularly preferred are pharmacologically active ingredients increasing the activity of Raf, in particular B-Raf, a modification of the survival rate of the cells of the central nervous system being a particularly preferred effect of the pharmacologically active ingredient(s). Preferably the pharmacologically active ingredients identified by the method of the present invention increase or inhibit the activity of B-Raf, not however that of C-Raf or A-Raf.

For identifying pharmacologically active ingredients which influence, preferably increase or inhibit, the activity of B-Raf, not however that of C-Raf, cells lacking the c-raf gene can be used for control. Such cells can for instance be obtained from c-raf (−/−) deficient mice (Wojnowski et al., Mech. Dev. 76:11-149, 1998).

Another subject matter of the present invention is a method for the in vitro analysis of the function of cells of the central nervous system, characterized by that the activity of Raf, in particular of B-Raf, IAP-1, IAP-2, x-IAP and/or survivin in the cells and/or cell extracts is determined. For this purpose, cells of the central nervous system were taken from the patient. These cells can now immediately be tested for the activity of the above proteins, either one of the above methods being used for the cell itself or for cell extracts obtained from the cell. Furthermore, it is possible to cultivate the cells isolated from the patient, and prior art methods can be employed for the cultivation of cells of the central nervous system. This is for instance desirable when the number of the isolated cells of the central nervous system is small and/or the analysis cannot immediately take place after removal of the cells. The cultivation permits to determine at a later time the activity of the above proteins either directly in the cells and/or cell extracts.

Another subject matter of the invention is a diagnostic substance for the in vitro analysis of the function of cells of the central nervous system, comprising at least one agent for the detection of the activity of Raf, in particular B-Raf, IAP-1, IAP-2, x-IAP and/or survivin.

The diagnostic substance according to the invention comprises for instance one or more DNA oligonucleotide pairs permitting the multiplication (PCR) of DNA fragments, in particular cDNA fragments coding for the proteins Raf, in particular B-Raf, IAP-1, IAP-2, x-IAP and/or survivin. A preferred diagnostic substance according to the invention comprises a DNA probe pair for detecting the activity of B-Raf and another probe pair for detecting the activity of A-Raf, C-Raf, IAP-1, IAP-2, x-IAP and/or survivin. Further diagnostic substances according to the present invention comprise for instance antibodies directed against Raf, in particular B-Raf, IAP-1, IAP-2, x-IAP, survivin, activated Raf, in particular activated B-Raf and/or a protein directly or indirectly activated by Raf, such as for instance MEK. A preferred subject matter of the diagnostic substance according to the invention consists of two antibodies selected from the above antibodies. Preferred combinations are here an antibody against B-Raf and against activated B-Raf, against activated B-Raf and IAP-1, IAP-2, x-IAP and/or survivin.

Another subject matter of the present invention is a test system for identifying pharmacologically active ingredients which influence the function of cells in a central nervous system. The test system comprises:

a) at least one sample, in particular at least one cell, at least one cell extract, at least one protein mixture and/or at least one mixture containing Raf, in particular activated B-Raf or a part thereof; and

b) at least one agent for determining the Raf activity, in particular the B-Raf activity.

In a preferred embodiment of the test system, the sample is compartmentalized, for instance on a microtiter plate with 96, 348 or 1,552 wells. Such microtiter plates are already used as routine in fully automatic, massive parallel test methods. Basically every compartmentalization is suitable which permits to spatially limit the effect of the potential active ingredient brought into contact with the sample, such that the effects of the respectively used potential active ingredient on the activity of Raf in the sample can be determined. The sample may be covalent or non-covalent with the surface of the sample carrier, such as for instance a microtiter plate, or may be present in a solution, a suspension or a flotation.

Another subject matter of the invention is a drug for treating diseases occurring with a disturbance of the function of the cells of the central nervous system, containing Raf, in particular B-Raf, and if applicable suitable auxiliary and additional substances. The drug may for instance contain Raf protein and/or DNA sections coding for Raf protein. Suitable auxiliary and additional substances are for instance protease inhibitors, detergents, buffers, viral vectors, such as for instance recombinant adenoviruses (Gravel et al., nature Med. 3:765-770, 1997), transfection reagents, such as for instance lipofectamines and substances with comparable mode of operation (Götz et al., Hum. Mol. Genet. 9:2479-2489, 2000) or buffer reagents for the transfer of expression vectors in cells with transient membrane permeabilization (Wiese et al., Nature Neurosci. 2:978-983, 1999).

The drug of the present invention is preferably used for disturbances of the function of cells of the central nervous system, which are characterized by a reduction of the survival rate of the cells, such as for instance cerebral ischemia (infarction), amyothrophic lateral sclerosis (ALS), Alzheimer's disease, nerve lesions, multiple sclerosis, Parkinson's disease, diabetic neuropathy, spinal muscular atrophy, prion diseases, such as for instance Creutzfeldt-Jakob disease (CJD).

A preferred drug of the present invention comprises Raf, in particular B-Raf, in a vector. The term vector in the meaning of the present invention relates to plasmid vectors, in particular episomal replicating vectors, viral vectors, and suitable viral vectors are for instance herpesviruses, adenoviruses, adeno-associated viruses, papillomaviruses or HIV1 or are derived from these viruses. The man skilled in the art knows about a series of further viruses which are in the same way suitable for the transfer of Raf protein, in particular B-Raf proteins and/or for the transfer of nucleic acids coding for Raf protein, in particular for B-Raf, such as for instance liposomes, virosomes, fusion proteins with e.g. antennapedia (Thoren et al., FEBS Lett. 482:265-268, 2000) or HIV-TAT (Arese et al., J. Immunol. 166:1380-1388, 2001).

The following examples are intended for a more detailed description of the invention only, however without limiting the same.

### EXAMPLES

1. Isolation and Cultivation of Neurons.

b-raf (+/−) heterozygous or c-raf (+/−) heterozygous parent animals were interbred back (Wojnowski et al., Mech. Dev. 76:141-149, 1998; Nature Genet. 16:293-297, 1997). From embryos of 12.5 days age same as from newborn mice which were homozygous for b-raf (−/−) or c-raf (−/−), spinal motoneurons were isolated by means of the panning technology (Metzger et al., J. Neurosci. 1735-1742, 1998) under utilization of a monoclonal rat anti-p75 antibody (Chemicon, Hofheim, Germany). For this purpose, the ventrolateral parts of the lumbal spinal cord were mechanically disintegrated, transferred into a Hepes buffer solution (containing 10 μM 2-mercaptoethanol) and incubated with trypsin (0.05%, 10 min). The individual cell suspension in the supernatant was transferred into a culture dish coated with the anti-p75 antibody and incubated at ambient temperature for 30 min.

Subsequently the individual culture dishes were washed, then the adhering cells were removed from the culture shell by a 0.8% saline solution containing 35 mM KCl and 1 μM 2-mercaptoethanol.

The thus obtained cells were sown at a density of 2,000 cells/cm² in culture plates (Greiner, Nürtingen, Germany) pre-coated with polyornithine and laminin. The cells were held at 37° C. in neurobasal medium (Life Technologies, with B27 supplement, 10% horse serum, 500 μM Glutamax and 50 μg/ml apotransferrin) and in a 5% CO₂ atmosphere. 50% of the cell culture medium were replaced on day 1 and subsequently every second day.

The analysis of the mRNA for IAP-1, IAP-2, x-IAP and t-IAP (survivin) was made by means of the RT-PCR. RNA was isolated using trizol reagent (Life Technologies, Karlsruhe), and 10 ng total RNA each were used for a TR-PTR reaction. The primer sequences for the amplification of IAP-1, IAP-2, x-IAP and t-IAP (survivin) were as follows: IAP-1f: 5′-TACTACATAGGACCTGGAGA-3′, IAP-1r: 5′-CCCACCATCACAGCAAAA-3′, annealing temperature: 55° C., IAP-2f: 5′-GGAGAAGAAAA TGCTGACCC-3′, IAP-2r: 5′-GCTTGTAAGGGTATCTGTGT3′, annealing temperature: 55° C., x-IAPf: 5′-TGC AAGAGCTGGATTTTATG-3′, x-IAPr: 5′-CCCGATCTGGC AGCTGTACC-3′, annealing temperature: 55° C.; tIAP (SURVIVIN), tIAPf: 5′-CCA GAT CTG GCA GCT GTA CC-3′ and tIAPr: 5′-GCC AGC TGC TCA ATT GAC TG-3′, annealing temperature: 64° C. As a control for the integrity of the RNA, part of the β-actin mRNA were amplified with the following primers: β-actinf: 5′-GTGGGCCGCC CTAGGCACCAG-3′, β-actinr: 5′-CTCTTTAATGTCACGCAC GATTTC-3′, annealing temperature: 64° C. The RT-PCR was performed following the protocol of the manufacturer with random hexamer primers. The PCR amplification was made as follows: 94° C., 30 sec, indicated annealing temperature, 1 min, 72° C., 1 min. IAP-1 and t-IAP (survivin) were treated for 33 and 35 cycles, IAP-2 and x-IAP for 28 and 30 cycles and β-actin for 26 and 28 cycles. The PT-PCR at RNA of E12.5 brains of b-raf and c-raf +/− pairings resulted in a distinct reduction by on the average 60% and 55% of IAP-1 for b-raf and c-raf −/− embryos in comparison to the wild-type control, 52% of IAP-2 for b-raf −/− and 46% of x-IAP for b-raf −/− embryos in comparison to the wild-type control.

From embryos 12.5 days age, same as from unborn mice, which were homozygous for b-raf (−/−) or c-raf (−/−), further sensor neurons were isolated. For this purpose, dorsal root ganglia were isolated, incubated for 30 min in PBS and with trypsin (0.05% in Hepes buffer). The trypsin digestion was stopped by addition of L15 medium containing 10% horse serum, and then the cells were plated out in culture plates for 3-4 hours. Cells in the supernatant were centrifuged (10 min 400 g) and the cell sediment was held, same as described for spinal motoneurons, in a neurobasal medium.

2. Isolation and Cultivation of Neuronal Stem Cells.

Neural stem cells were isolated from the brain of normal b-raf (−/−) or c-raf (−/−) deficient mouse embryos as well as from newborn mice. The zone of the forebrain is removed under a preparation microscope, in further developed embryos also the zone of the hippocampus and the periventricular zone. These brain areas were then transferred in 200 μl HBSS (Hanks balanced salt solution (HBSS), Life Technologies, Karlsruhe), incubated with 0.1% trypsin (final concentration in HBSS) for 10 min at 37° C., the reaction was stopped with 0.1% trypsin inhibitor (trypsin inhibitor from egg yolk sack (Sigma, Deisenhofen), stock solution: 1% in HBSS/25 mM HEPES) final concentration in HBSS. Then the cells were triturated 10 times with a 200 μl pipette and transferred in medium [(Neurobasal medium (Life Technologies), B27 supplement (Life Technologies stock 50×, EK 1×) Glutamax II (Life Technologies stock 10×, EK 1×), basicFGF (20 ng/ml), EGF (20 ng/ml)I] in a volume of 5 ml. The dissociated cells were cultivated in Sarstedt dishes (50 ml) (breeding chamber, 37° C., 5% CO₂, atmosphere saturated humidity), the medium was changed every two days. The cells grow as embroid bodies and do not attach, thus the cells are transferred for the medium change into a Falcon tube and centrifuged for 5 min at 400 g. The supernatant is sucked off, and the cell sediment is titruated and received in fresh medium. At the latest after 3 passages, large embroid bodies will form which can be trypsinated (see above) and plated at a low cell density (max. 10,000 cells/plate) on 10 cm dishes (Sarstedt). Individual cells are then picked and firstly expanded in 96 well plates, later in 24 and 12 well plates. These individual cell clones of neural stem cells can then be tested for their differentiation capacity and then used in the test methods.

In order to obtain reproducible results, the cells were also established as lines and deep-frozen and stored for later experiments.

Deep-freezing of the neural stem cells follows a standard protocol, i.e. after centrifugation, the cells were received in a medium with 10% DMSO and firstly cooled down with 1° C./min to −86° C. (in the MrFrosti), and then stored in liquid N₂ at −186° C.

3. Effects of Neurotrophic Factors on b-raf (−/−) Deficient Neurons.

To motoneurons were added as neurotrophic factors GDNF, BDNF and CNTF (1 ng/ml each), and NGD (1 ng/ml) to the sensor neurons. In the control cultures of normal mice, only approx. 10-25% of the cells survived without addition of the respective neurotrophic growth factors, with addition of the respective growth factors however survived approx. 70% of the originally sown neuronal cells. Whereas neuronal cell cultures of c-raf (−/−), c-raf (+/−) or b-raf (+/−) deficient embryos or mice have in this regard no difference to cultures of normal mice, no effects of neurotrophic factors on the survival of motoneurons or sensor neurons of b-raf (−/−) deficient embryos or newborn mice could be detected. For b-raf (−/−) deficient neurons the survival rate after 3 days cell culture with or without neurotrophic factors was at figures smaller than 3% of the sown neurons.

These results show that B-Raf is a decisive signal-transducing protein for the survival of sensor and motor nerve cells.

4. Method for Identifying Substances Which Protect Nerve Cells.

Thus nerve cells which are obtained for instance by the above method can be used for the search of substances protecting nerve cells from a cell death.

For this purpose, b-raf (−/−), b-raf (+/−), c-raf (−/−) deficient and normal motor neurons and sensor neurons as described above are obtained, sown in cell cultures and reacted with the test substance. Substances which protect nerve cells are capable to prevent the death of b-raf (−/−) neurons without impairing the survival of b-raf (+/−), c-raf (−/−) or normal neurons.

For a model-type test of the method, sensor neuronal cells were transfected with a plasmid (pCDNA3) which contained the open reading frame of the B-Raf gene (Wojnowski et al. Mech. Dev. 91:97-104, 2000) or with a LacZ expression plasmid by the method specified by Wiese et al. (Nature Neurosci. 2:987-983, 1999), and the survival of the thus transfected neurons in the cell culture was determined.

Whereas b-raf (−/−) deficient neurons being not transfected or being transfected with the LacZ expression plasmid died in cell culture with and without neurotrophic factors, b-raf (−/−) deficient neurons being transfected with the plasmid containing the b-raf gene survived. Immunofluorescence investigations of surviving b-raf (−/−) deficient neuronal cells having been transfected with a plasmid containing the b-raf gene had the result, with an antibody being specific for the B-Raf protein (Sithanandam et al., Oncogene 5:1775-1780, 1990), that these cells contain the B-Raf protein.

These investigations show that by a suitable active ingredient (plasmids coding for B-Raf) b-raf (−/−) deficient neuronal cells can be protected from death, this method thus being suitable for identifying substances which protect nerve cells.

5. Testing Active Ingredients.

The following active ingredients were tested with the method according to the invention:

GW 5074, inhibitor of the C-Raf kinase (IC50 value of 9 nM; Lackey et al. Bioorg. Med. Chem. Lett. 10:223 (2000)

EMD 400073; inhibitor of the B-Raf kinase (IC50 value of 1 μM; Boehringer Pharmacology Congress 2001)

ZM 338372; inhibitor of the C-Raf kinase (IC50 value of 70 nM; Hall-Jackson et al Chem Biol. 6:559 (1999)

The substances were added at concentrations of 0.1; 1.0; 10 and 100 μM to the motoneurons held in the culture with and without CNTF (1 ng/ml), and the number of the apoptotic and of the surviving cells were counted after 24 hours. Whereas the B-Raf inhibitor caused a strong apoptosis with as well as without CNTF, in the cell cultures treated with C-Raf inhibitors survived as many cells as in the respective control groups to which no inhibitor was added.

These results show that the specific inhibition of B-Raf will lead to an apoptosis of neurons, whereas in contrast thereto an inhibition of C-Raf does not influence the survival of neurons.

6. Testing Active Ingredients for the Regeneration of Nerves.

In adult mice, the nervus facialis was cut off under Ketanest/Rompun anesthesia (100 mg/kg), and locally at the cut-off nerve GW 5074 and EMD 400073 active ingredients of 20 μM each were applied on a gel foam piece and brought in place at the distal nerve stump. In a control group, the nerve was severed, and on the gel foam piece was applied the solvent (100% DMSO). The following result is obtained: An investigation of the animals after 14 days will yield a 90% survival for the control group, since the motoneurons of the nervus facialis in the adult mouse will react again after the severance of the axon. The application of GW 5074 should not be a disadvantage for the regeneration capability of the motoneurons, whereas the application of EMD 400073 should lead to a distinct motoneuron loss. (Saturating amounts of the respective substances were used, since a titration in vivo is not possible due to the unclear absorption by the surrounding tissue.)

1. A method for identifying pharmacologically active ingredients which influence the function of cells in a central nervous system, comprising the following steps:
a) a sample is brought into contact with at least one potential active ingredient, and
b) the activity of B-Raf in the sample is determined.

2. A method according to claim 1, wherein the sample contains at least one cell, at least one cell extract, at least one protein mixture and/or one mixture containing B-Raf, in particular activated B-Raf, or a part thereof.

3. A method according to claim 2, wherein the cell is a glial cell; a neuronal cell, for instance a sensor, sympathic or motor neuronal cell; a neuronal stem cell; a neuron, in particular a cholinergic neuron of the basal forebrain, a dopaminergic nerve cell of the midbrain, a granule cell, a Purkinje cell of the cerebellum or the hippocampus; a retinal ganglion cell or a photoreceptor.

4. A method according to one of claims 2 or 3, wherein the cell has a reduced B-Raf activity or no B-Raf activity at all.

5. A method according to one of claims 1 to 4, wherein the activity of B-Raf in the cell is determined by the change of the survival rate of the cell.

6. A method according to one of claims 1 to 4, wherein the activity of B-Raf in the sample is directly or indirectly determined by the amount of the B-Raf protein, the amount of the nucleic acids coding for B-Raf and/or the enzymatic activity of B-Raf.

7. A method according to one of claims 1 to 6, wherein in a further step the activity of IAP-1, IAP-2, x-IAP and/or survivin is determined.

8. A method according to one of claims 1 to 7, wherein in a further step the pharmacologically active ingredient is isolated.

9. An active ingredient identified by the method according to one of claims 1 to 8.

10. A method for the in vitro analysis of the function of cells of the central nervous system, characterized by that the activity of B-Raf, IAP-1, IAP-2, x-IAP and/or survivin in the cells and/or cell extracts is determined.

11. A diagnostic substance for the in vitro analysis of the function of cells of the central nervous system, comprising at least one agent for the detection of the activity of B-Raf, IAP-1, IAP-2, x-IAP and/or survivin.

12. A test system for identifying pharmacologically active ingredients which influence the function of cells in a central nervous system, comprising:
a) at least one sample; and
b) al least one agent for determining the B-Raf activity in the sample.

13. A drug for treating diseases occurring with a disturbance of the function of the cells of the central nervous system, containing B-Raf and if applicable suitable auxiliary and additional substances.

14. A drug according to claim 13, wherein the disturbance of the function of the cells of the central nervous system is characterized by a reduction of the survival rate of the cells.

15. A drug according to claim 13 or 14, wherein B-Raf is included in a vector.
